(19) United States
(12) Patent Application Publication
(10) Pub. No.: US 2007/0038200 A1
(43) Pub. Date: Feb. 15, 2007

(54) Disposable-hygiene protective pouch

(76) Inventor: Tiffany Lynn Hill, Dallas, TX (US)

Correspondence Address: Tiffany Lynn Hill, 5839 Chapelwood Way, Dallas, TX, 75228, US

(21) Appl. No.: 11/498,453

(22) Filed: Aug. 2, 2006

Related U.S. Application Data
(60) Provisional application No. 60/706,874, filed on Aug. 10, 2005.

(51) Int. Cl. A61F 13/15

(52) U.S. Cl. 604/385.13; 604/359; 604/385.02

(57) ABSTRACT

The method of making a Disposable Hygiene Protective Pouch, includes the scented layer of material that is inserted on the outside of the waterproof plastic liner. The inner liner that is waterproof guards against leakage. An additional lining is the outer layer of material which provides protection against punctures and tears.

### BACKGROUND OF INVENTION

1. Field of Invention

This invention relates to a Disposable Hygiene Protective Pouch from which odors are eliminated and neutralized. More specifically my invention becomes a portable container system while on the go.

2. Discussion of the Related Art

Numerous scented plastic bags have been provided in prior art. Several types of thin plastic scented bags have been proposed. Although inexpensive to manufacture, if the scented bag becomes punctured or torn the scented plastic bag is useless and stands a chance of the odor escaping and also leakage may occur. These plastic scented bags simply do not possess the creativity and effective method of containing odors.

It is also known that the scented plastic bags with or without tie handles, after being shifted around may become loose. They do not have the security of always guaranteeing odors that should be properly contained, therefore, causing the odor to escape into the environment. Unfortunately, these scented plastic bags have no protective seal to ensure leakage, or odors, and guarding against any punctures or tears. They are simply not designed with the uniqueness of masking odors. However, they do not provide security for the consumer, making the bags useless and a non-effective method of discreetly keeping a personal hygiene or diaper need, safe and secure.

### BRIEF SUMMARY OF THE INVENTION

In response to the difficulties and problems discussed above a new Disposable Hygiene Protective Pouch has been designed.

The method comprises putting the desired layers of materials into the Disposable Hygiene Protective Pouch. An inner plastic waterproof panel includes a fluid absorbent core, longitudinally positioned along the length of the pouch which is circumscribed by a plurality of fused sides. Another outer liner that covers the inner plastic liner is a scented layer of material to neutralize and eliminate odors while keeping anything that is soiled in the pouch discreet and lightly scented. Again the pouch provides a protective layer that becomes a barrier against punctures or tears making it difficult for rips that could cause an odor to be released, or leakage that may damage the pouch, and or be exposed.

Lastly the adhesive seal on the flap, which closes the pouch to its entirety, makes It even more complicated to be reopened for exposure. It now becomes a easy, portable Container, which is safe and secure until properly disposed of.

### BRIEF DESCRIPTION OF THE DRAWINGS

FIG. 1 Shows a laid open Disposable Hygiene Protective pouch with the various parts Thereof.

FIG. 2 Is a schematic front view of an opened pouch.

FIG. 3 Is a schematic side view of a pouch.

FIG. 4 Illustrates a view for accomplishing the closing of the pouch.

FIG. 5 Illustrates a complete secured pouch closed.

FIG. 6 & 7 Illustrates examples of various sizes and designs for the pouch.

### DETEAILED DESCRIPTION FO THE PRESENTLY PREFERRED EMBODIMENTS

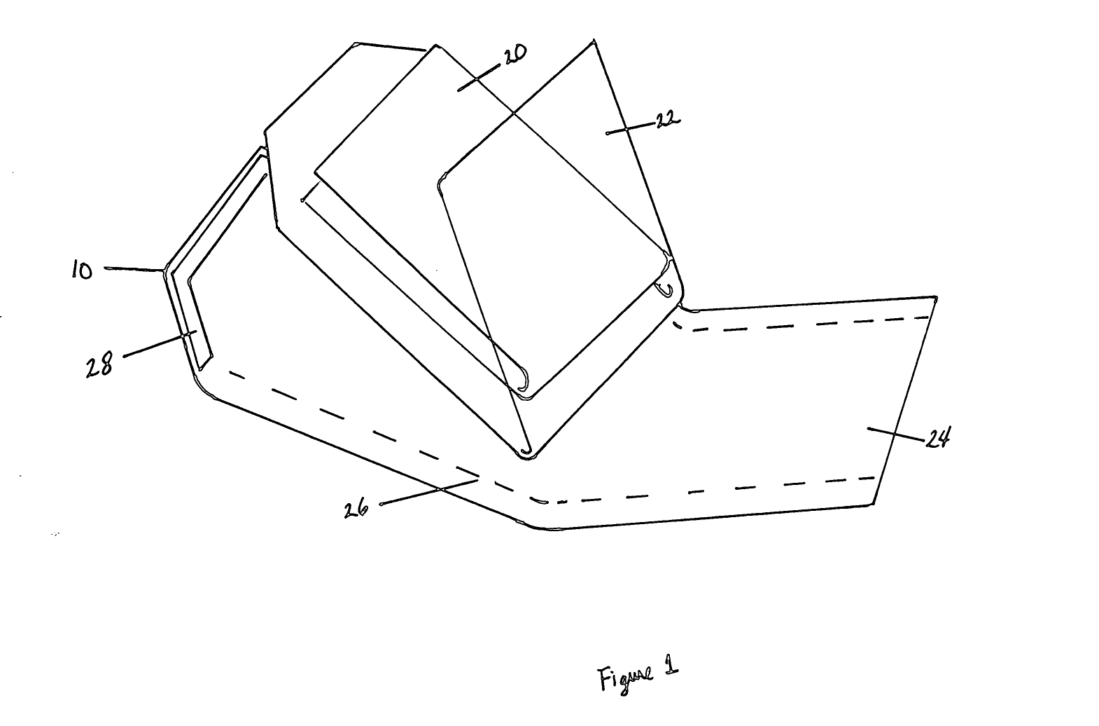

With reference to FIG. 1, 20 or plastic inner absorbent core which is substantially a waterproof liner Inserted between 22 and 24 with 22 being the layer of material that is scented, 20 is covered over the 20 plastic liner. The 20 plastic liner is fused to 22, a layer more durable which is 24, goes over 22, to help store and lock in 22, scented layer and now becomes fused to 24, along the sides of 26, to make the pouch. The adhesive strip 28 is added to the top inside edge of the closure flap of the pouch.

FIG. 1 is a representative plan view of the pouch of the present invention in a flat, uncontracted state. Portions of the structure are partially cut away to more clearly show the interior construction of the pouch. 24, and the surface of the pouch are facing the viewer. The pouch inner lining 20, includes a waterproof plastic liner which is vapor permeable that acts as an absorbent core 20, positioned in facing relation with the back sheet 22, which is located between the back sheet and the top sheet 24. The pouch 26, has a lateral direction and a longitudinal direction. Marginal portions of the pouch 26, such as the back sheet protective layer 24, may extend past the terminal edges of the scented layer 22. In the illustrated embodiment, for example, the back sheet protective layer 24, extends outwardly beyond the marginal edges of the scented layer 22, to form side margins, 26, and end margins of the pouch. The top sheet (exterior) 24, is generally coextensive with the back sheet scented layer 22, but may cover an area which is larger or smaller than the area of the back sheet (exterior) 24, as desired.

To provide improved fit to help reduce leakage of body exudates from the pouch 20, the side margins 26 and 22 side edges are thermo bonded to hold together and seal the pouch.

The pouch, as representatively illustrated in FIG. 1, may further include a protective sealant strip 28, illustrated by an adhesive glue strip to 24, top inside edge (for flap closure). A suitable adhesive tape fastener 28, is added to make a properly closed and protective seal. It is lightly bonded to the top inside edge of the flap closure to provide a more secure and closed pouch when sealed.

The various components of the pouch are assembled together employing various types of suitable attachment means, such as thermal bonds, adhesives, or combination thereof.

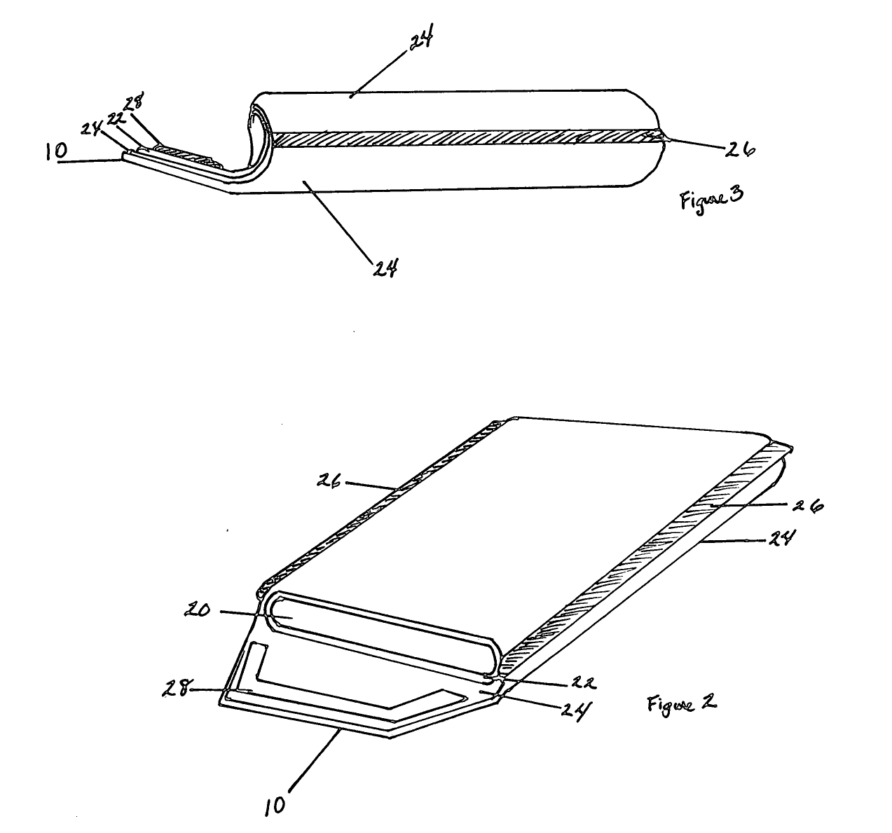

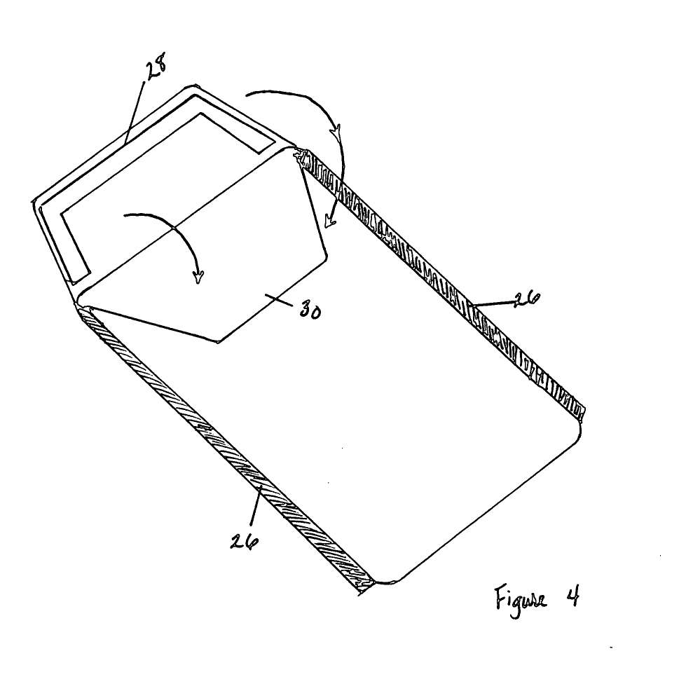

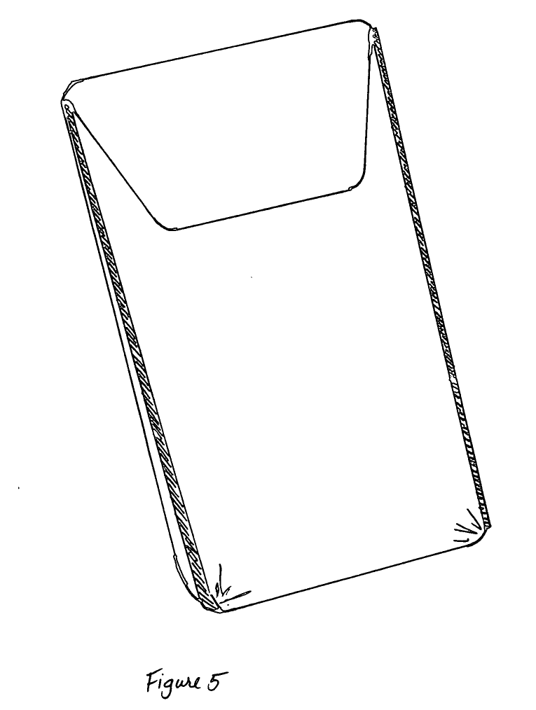

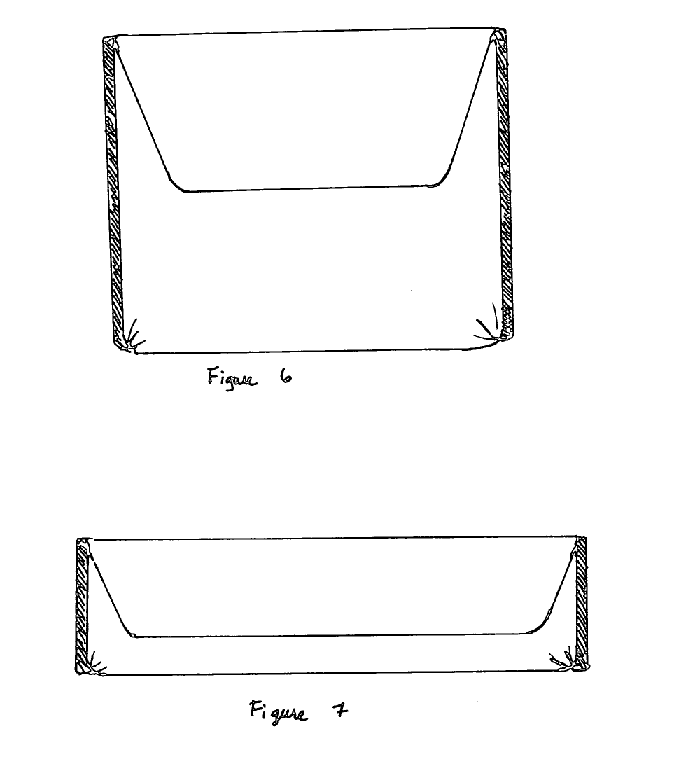

Referencing FIG. 2 and FIG. 3, the pouch 10, is in a folded and fastened state whereby the cooperating fastening system 26, of the front and side view of the pouch. The pouch 10, has fastened seamed side panels which have been registered by folding the laid open pouch of FIG.1. Each layer folded over to a medial line which does not cover flap closure (top of 24) brings the respective interior and exterior layers to mating registration and fastening them 26.

While in the foregoing specification this invention has been described in relation to certain preferred embodiments thereof, and many details have been set forth for the purpose do illustration, it will be apparent to those skilled in the art that the invention is susceptible to additional embodiments.

What I claim my invention is:

1. A Disposable and protective material comprising a mixture of scent to eliminate and neutralize odors, and a inner lined liquid resistant pocket that interstices between the scented and protective material, whereby a universal pouch has means for the use of body exudates and proper disposal of waste, wherein a convenient, portable pouch will be provided.
